(19) United States
(12) Patent Application Publication
(10) Pub. No.: US 2021/0169870 A1
(43) Pub. Date: Jun. 10, 2021

(54) USES OF FERMENTATION PRODUCT OF LOTUS LEAF-HAWTHORN EXTRACT AND ITS ACTIVE INGREDIENT IN INHIBITING THE FORMATION OF FAT

(71) Applicant: TCI Co., Ltd., Taipei (TW)

(72) Inventors: Yung-Hsiang LIN, Taipei (TW); Wei-Hsiu CHUANG, Taipei (TW)

(21) Appl. No.: 16/781,822

(22) Filed: Feb. 4, 2020

(30) Foreign Application Priority Data
Dec. 4, 2019 (TW) 108144300

(51) Int. Cl. A61K 31/473; A61K 36/734

(52) U.S. Cl. CPC A61K 31/473; A61K 2236/331; A61K 2236/19; A61K 36/734

(57) ABSTRACT

Use of at least one of the following compounds of formula (I) to formula (III) is provided:

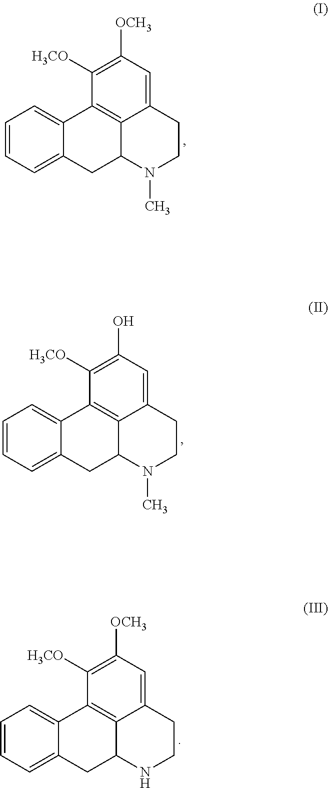 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

Provided is a composition comprising a fermentation product of lotus leaf-hawthorn extract that can provide at least one of the above compounds of formula (I) to formula (III), and also provided is a method for preparing the fermentation product of lotus leaf-hawthorn extract.

### CROSS REFERENCE TO RELATED APPLICATIONS

This application claims priority to Taiwan Patent Application No. 108144300 filed on Dec. 4, 2019, in the Taiwan Intellectual Property Office, the disclosure of which is incorporated herein in its entirety by reference.

### FIELD OF THE INVENTION

The present invention relates to the uses of at least one of the compounds of formula (I) to formula (III), especially relates to the uses of those compounds in at least one of promoting the lipid-degradation ability of adipocyte, inhibiting the lipid-forming ability of adipocyte, making the formation of body fat difficult and promoting weight loss:

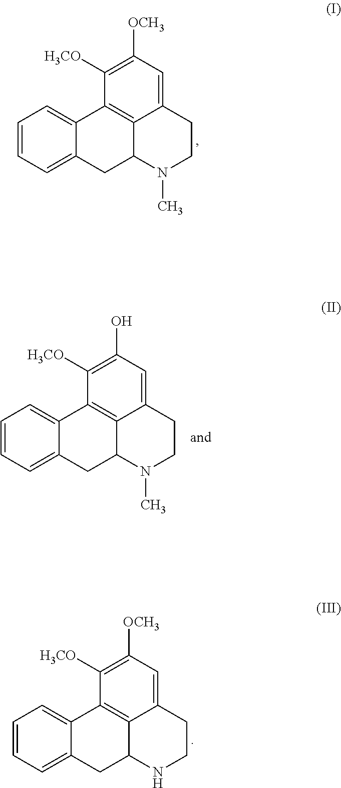 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

The present invention also relates to a composition comprising a fermentation product of lotus leaf-hawthorn extract that can provide at least one of the above compounds of formula (I) to formula (III), and a method for manufacturing the fermentation product of lotus leaf-hawthorn extract.

### BACKGROUND OF THE INVENTION

Obesity is a condition in which the body of an organism contains excess fat. The usual international indicators used to evaluate obesity in humans are body mass index (BMI) or waistline. In Taiwan, if the BMI value of a person is greater than or equal to 24 and smaller than 27, it means the person is overweight; if the BMI value of a person is greater than or equal to 27, it means the person is obese.

In view of high-fat and high-sugar diets along with insufficient exercise, many people in modern society face the problem of obesity. Obese people have a higher incidence of metabolic and cardiovascular diseases such as diabetes, fatty liver, hyperlipidemia, hypertension, heart disease and stroke. They even are at more risk of developing knee arthritis and gout, and thus, obesity may cause a severe threat to the physical health at last. There is also research showing that obesity is an important carcinogenic factor. Currently, the World Health Organization has considered obesity as a chronic disease. Besides, people who are suffering from obesity have a greater likelihood of developing psychological problems and encountering social communication disturbances. Therefore, in recent years, medical research has focused on finding methods to inhibit obesity, and in turn, promote physical and mental health.

For obese people, the common methods for inhibiting obesity over recent years include diet control, exercise, life-style changes, medicinal treatments, and surgery. Except for the severely obese patients who may need surgery, the usual recommended clinical methods for losing fat are diet control and exercise. However, diet control requires a person to strictly balance his/her diet and control the caloric intake, and thus, it is hard to practice. Exercise may also cause body injury if not done appropriately. In addition, because both of the above methods do not directly target the adipocytes, those methods only have a limited effect in helping one to lose fat (especially fat around the viscera).

Furthermore, due to the demand of maintaining good posture and the preference of aesthetic perception in modern society, even people with normal BMI values and waistlines (i.e., people who are not clinically obese) desire to lose fat or weight to improve their appearance, or wish to make the formation of body fat difficult by consuming health food. Therefore, there is a need to develop a composition that is convenient to use and effective in reducing the accumulation of fat.

The inventors of the present invention found that the following compounds of formula (I) to formula (III) are effective in reducing the accumulation of fat:

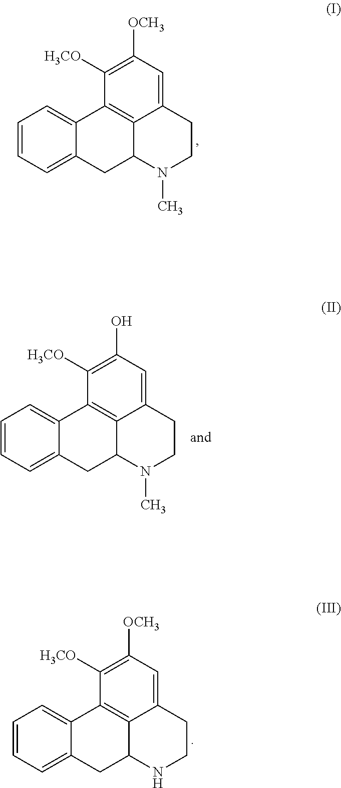 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

The inventors of the present invention also found that fermenting a lotus leaf-hawthorn extract can increase the content of at least one of the above compounds of formula (I) to formula (III) in the fermentation product of lotus leaf-hawthorn extract. Therefore, the present invention also relates to a composition comprising the fermentation product of lotus leaf-hawthorn extract, and a method for manufacturing the fermentation product of lotus leaf-hawthorn extract.

### SUMMARY OF THE INVENTION

An objective of the present invention is to provide a use of an active ingredient in the manufacture of a pharmaceutical composition, wherein the pharmaceutical composition is for at least one of promoting the lipid-degradation ability of adipocyte, inhibiting the lipid-forming ability of adipocyte and promoting weight loss, and the active ingredient is at least one of the following compounds of formula (I) to formula (III):

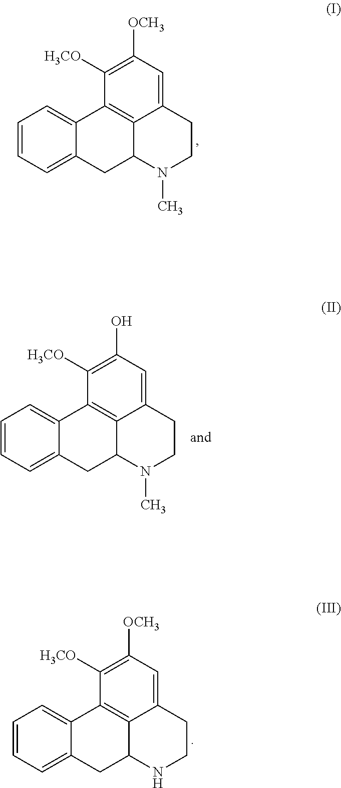 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

Another objective of the present invention is to provide a use of an active ingredient in making the formation of body fat difficult, wherein the active ingredient is at least one of the following compounds of formula (I) to formula (III):

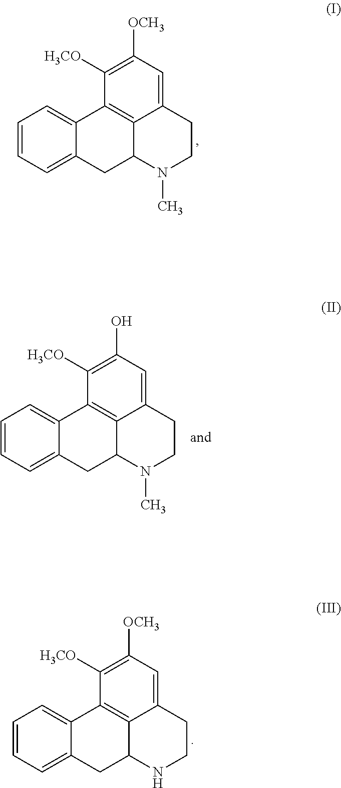 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

The active ingredient can be used in the form of a food composition.

Still another objective of the present invention is to provide a method for at least one of promoting the lipid-degradation ability of adipocyte, inhibiting the lipid-forming ability of adipocyte, making the formation of body fat difficult and promoting weight loss, the method comprising administering to a subject in need an effective amount of an active ingredient, wherein the active ingredient is at least one of the compounds of formula (I) to formula (III):

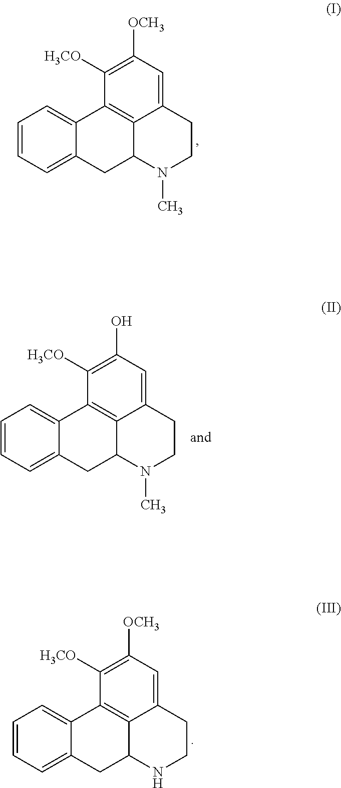 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

Preferably, the above active ingredient is provided in the form of a fermentation product of an extract. More preferably, the extract is a lotus leaf-hawthorn extract.

Yet another objective of the present invention is to provide a composition for at least one of promoting the lipid-degradation ability of adipocyte, inhibiting the lipid-forming ability of adipocyte, making the formation of body fat difficult and promoting weight loss, the composition comprising a fermentation product of lotus leaf-hawthorn extract. The composition can be a pharmaceutical composition or a food composition.

Yet another objective of the present invention is to provide a method for manufacturing the above fermentation product of lotus leaf-hawthorn extract, the method comprising the following steps:
(a) mixing lotus leaf and hawthorn to provide a mixture;
(b) extracting the mixture to provide an extract;
(c) fermenting the extract by using Saccharomyces cerevisiae and Lactobacillus sp. strains to provide an intermediate fermentation product; and
(d) fermenting the intermediate fermentation product by using an Acetobacter sp. strain to provide a fermentation product of lotus leaf-hawthorn extract.

Preferably, the weight ratio of lotus leaf and hawthorn in step (a) is 1:1 to 3:1. More preferably, the extraction in step (b) is performed by using water that optionally contains saccharide.

The detailed technology and preferred embodiments implemented for the present invention are described in the following paragraphs for people skilled in this field to well appreciate the features of the claimed invention.

### BRIEF DESCRIPTION OF THE DRAWINGS

The patent application contains at least one drawing executed in color. Copies of this patent with color drawings will be provided by the Patent and Trademark Office upon request and payment of the necessary fee.

FIGS. 1A to 1C show the NMR spectrums, wherein FIG. 1A shows the NMR spectrum of the compound of formula (I), FIG. 1B shows the NMR spectrum of the compound of formula (II), and FIG. 1C shows the NMR spectrum of the compound of formula (III).

FIGS. 2A to 2C show the photos of adipocytes dyed with oil-red O dye that were taken by a microscope, wherein FIG. 2A shows the cells that were not treated with the analyte, FIG. 2B shows the cells that were treated with the lotus leaf-hawthorn extract, and FIG. 2C shows the cells that were treated with the fermentation product of lotus leaf-hawthorn extract, wherein the scale represents 500 μm.

FIG. 3 is a bar chart expressing the relative fat content of each group, wherein “group I” was cultured in a medium free of analyte, “group II” was cultured in a medium containing the lotus leaf-hawthorn extract, and “group III” was cultured in a medium containing the fermentation product of lotus leaf-hawthorn extract.

FIG. 4 is a bar chart expressing the relative fat content of each group, wherein “group i” was cultured in a medium free of analyte, “group ii” was cultured in a medium containing the fermentation product of lotus leaf-hawthorn extract, “group iii” was cultured in a medium containing the n-butanol layer extract of the fermentation product of lotus leaf-hawthorn extract, and “group iv” was cultured in a medium containing the water layer extract of the fermentation product of lotus leaf-hawthorn extract.

FIG. 5 is a bar chart expressing the relative fat content of each group, wherein “group A” was cultured in a medium free of analyte, “group B” was cultured in a medium containing the compound of formula (I), “group C” was cultured in a medium containing the compound of formula (II), and “group D” was cultured in a medium containing the compound of formula (III).

FIG. 6 shows the HPLC fingerprints, wherein the top figure shows the HPLC fingerprint of the fermentation product of lotus leaf-hawthorn extract, and the bottom figure shows the HPLC fingerprint of the lotus leaf-hawthorn extract.

FIG. 7 is a bar chart of the subjects' body weights at week 0 and week 4.

FIG. 8 is a bar chart of the subjects' waistlines at week 0 and week 4.

FIG. 9 is a bar chart of the subjects' body mass indexes (BMI) at week 0 and week 4.

FIG. 10 is a bar chart of the subjects' body fat percentages at week 0 and week 4.

FIG. 11 is a bar chart of the subjects' trunk fat percentages at week 0 and week 4.

FIG. 12 is a bar chart of the subjects' evaluation scores of the level of obesity of themselves at week 0 and week 4.

### DESCRIPTION OF THE PREFERRED EMBODIMENTS

The following describes some of the embodiments of the present invention in detail. However, without departing from the spirit of the present invention, the present invention may be embodied in various embodiments and should not be limited to the embodiments described in the specification or defined in the appended claims.

Unless otherwise indicated herein, the expressions “a,” “an,” “the,” or the like recited in the specification of the present invention (especially in the claims) are intended to include both the singular and plural forms. The term “subject” recited in this specification refers to a mammalian, including human and non-human animals.

The term “lotus leaf” recited in this specification refers to the leaf of Nelumbo nucifera (also called lotus). Nelumbo nucifera is a perennial aquatic-herb belonging to the Nelumbonaceae family and Nelumbo genus, which is widely distributed throughout the world. The roots of Nelumbo nucifera are embedded in the sludge under water, while its leaf floats on the surface of water. The lotus leaf has a circular shape, with the diameter being about 60 cm. All of the flowers, seeds, leaves, roots and stems of Nelumbo nucifera are edible.

The term “hawthorn” recited in this specification refers to the fruit of plants belonging to the Rosaceae family, Maloideae subfamily, Crataegus spp., wherein Crataegus spp. comprises about 200 species. Hawthorn is a shrub or arbor, and its fruit usually is red in color and is edible. In a preferred embodiment, the Crataegus spp. used in this specification is Crataegus pinnatifida.

The term “lotus leaf-hawthorn extract” recited in this specification refers to the extract obtained by extracting the leaves of lotus and the fruit of hawthorn.

The terms “Saccharomyces cerevisiae,” “Lactobacillus sp. strain” and “Acetobacter sp. strain” recited in this specification comprise the Saccharomyces cerevisiae, Lactobacillus sp. strain and Acetobacter sp. strain that can be easily obtained by ordinary people (for example, those strains can be purchased from domestic and international deposit agencies), as well as the Saccharomyces cerevisiae, Lactobacillus sp. strain and Acetobacter sp. strain that can be isolated from natural sources by conventional isolation methods in this art.

The inventors of the present invention surprisingly found that all of the following compounds of formula (I) to formula (III) have the effect of reducing the fat content of adipocyte:

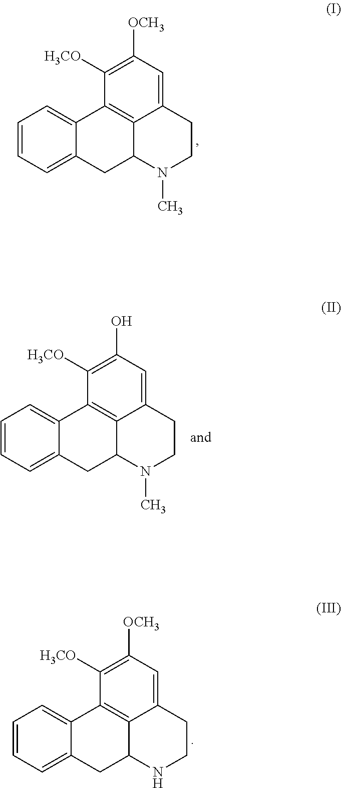 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

Therefore, the present invention provides a use of the above active ingredient in the manufacture of a pharmaceutical composition for at least one of promoting the lipid-degradation ability of adipocyte, inhibiting the lipid-forming ability of adipocyte and promoting weight loss; a use of the above active ingredient in the manufacture of a food composition for making the formation of body fat difficult; a use of the above active ingredient in making the formation of body fat difficult, and a method of administering an effective amount of the above ingredient to a subject in need.

As shown in the appended examples, according to the present invention, a lotus leaf-hawthorn extract can be obtained by extracting a mixture of lotus leaf and hawthorn, wherein the lotus leaf-hawthorn extract can be subjected to a fermentation process, to thus provide a fermentation product of lotus leaf-hawthorn extract containing the above compounds of formula (I) to formula (III). Therefore, at least one of the compounds of formula (I) to formula (III) according to the present invention can be provided in the form of a fermentation product of extract. In one preferred embodiment of the present invention, the extract is a lotus leaf-hawthorn extract. Additionally, the present invention also provides a composition for at least one of promoting the lipid-degradation ability of adipocyte, inhibiting the lipid-forming ability of adipocyte, making the formation of body fat difficult and promoting weight loss, the composition comprising the fermentation product of lotus leaf-hawthorn extract. In one preferred embodiment of the present invention, the fermentation product of lotus leaf and hawthorn can reduce the fat content of adipocyte when its concentration is at least 0.1% (w/w).

According to the present invention, the fermentation product of lotus leaf-hawthorn extract can be a fermentation product provided by the process comprising the following steps:
(a) mixing lotus leaf and hawthorn to provide a mixture;
(b) extracting the mixture to provide an extract;
(c) fermenting the extract by using Saccharomyces cerevisiae and Lactobacillus sp. strains to provide an intermediate fermentation product; and
(d) fermenting the intermediate fermentation product by using an Acetobacter sp. strain to provide a fermentation product of lotus leaf-hawthorn extract.

In step (a), the ratio of the amounts of the lotus leaf and hawthorn can be optionally adjusted. In general, there is no particular limitation to the ratio of the amounts of the lotus leaf and hawthorn being used. For example, in step (a), the lotus leaf and hawthorn can be used at a weight ratio ranging from 1:1 to 3:1 (lotus leaf:hawthorn).

In step (b), the extraction is performed by using water that optionally contains saccharide. The ratio of the amounts of the solvent and lotus leaf-hawthorn can be optionally adjusted. In general, there is no particular limitation to the amount of solvent being used, as long as the materials can be dispersed in the solvent evenly. For example, in step (b), the lotus leaf-hawthorn and solvent can be used at a weight ratio ranging from about 2:85 to about 4:65 (lotus leaf-hawthorn:solvent).

In step (b), the saccharide optionally contained in the solvent is at least one of monosaccharide, disaccharide and polysaccharide. In general, there is no particular limitation to the amount of the saccharide being used. For example, in step (b), the lotus leaf-hawthorn, saccharide and solvent can be used at a weight ratio ranging from about 2:7:91 to about 6:14:80 (lotus leaf-hawthorn:saccharide:solvent).

In step (b), the extraction can be conducted for a suitable period of time and at a suitable temperature depending on the selection of the solvent. When purified water is used as the solvent and the weight ratio of lotus leaf-hawthorn:solvent ranges from 2:85 to 4:65, the extraction is usually conducted at 50° C. to 100° C. (preferably at 70° C. to 85° C.) for 0.5 hour to 3 hours.

In step (c), Saccharomyces cerevisiae and Lactobacillus sp. strains are added into the extract provided in step (b) to conduct a first fermentation. In one particular embodiment of the present invention, Saccharomyces cerevisiae BCRC 20271 and Lactobacillus plantarum BCRC 910760 are selected to conduct step (c).

In step (c), the fermentation can be conducted for a suitable period of time and at a suitable temperature depending on the selection of the Saccharomyces cerevisiae and Lactobacillus sp. strains. When Saccharomyces cerevisiae BCRC 20271 and Lactobacillus plantarum BCRC 910760 are used, the fermentation is usually conducted at about 25° C. to about 35° C. for about 1 day to about 3 days.

In step (c), the amounts of Saccharomyces cerevisiae and Lactobacillus sp. strains can be optionally adjusted depending on the selection of the above two strains. When Saccharomyces cerevisiae BCRC 20271 and Lactobacillus plantarum BCRC 910760 are used, the amount of Saccharomyces cerevisiae BCRC 20271 is usually about 0.01 wt. % to about 0.5 wt. % of the extract; and the amount of Lactobacillus plantarum BCRC 910760 is usually about 0.01 wt. % to about 0.25 wt. % of the extract.

In step (d), an Acetobacter sp. strain is added into the intermediate fermentation product provided in step (c) to conduct a second fermentation. In one particular embodiment of the present invention, Acetobacter aceti BCRC 11688 is selected to conduct step (d).

In step (d), the fermentation can be conducted for a suitable period of time and at a suitable temperature depending on the selection of the Acetobacter sp. strain. When Acetobacter aceti BCRC 11688 is used, the fermentation is usually conducted at about 25° C. to about 35° C. for about 5 days to about 15 days.

In step (d), the amount of Acetobacter sp. strain can be optionally adjusted depending on the selection of the strain. When Acetobacter aceti BCRC 11688 is used, the amount of Acetobacter aceti BCRC 11688 is usually about 1 wt. % to about 20 wt. % of the intermediate fermentation product.

The fermentation product of lotus leaf-hawthorn extract provided in step (d) has the following characteristics: a sugar degree ranging from 35° to 45°, a pH value ranging from 3 to 5, and an alcohol concentration less than 3% (w/v).

Additionally, the fermentation product of lotus leaf-hawthorn extract provided in step (d) can be optionally subjected to extra operations such as vacuum concentration, filtration, and sterilization. For example, the fermentation product of lotus leaf-hawthorn extract of the present invention can be vacuum concentrated at 45° C. to 70° C. to provide a concentrated fermentation product. The fermentation product of lotus leaf-hawthorn extract of the present invention can also be filtrated by using a mesh screen with 200 to 400 meshes to remove the solid residues. Furthermore, before the fermentation product is administered to a subject, isomaltooligosaccharides can be optionally added to the fermentation product of lotus leaf-hawthorn extract in an amount of about 40% to about 70% (w/w). The fermentation product is sterilized at about 95° C. to about 120° C. for about 70 minutes to about 90 minutes to provide a beverage suitable for drinking.

As set forth above, the fermentation product of lotus leaf-hawthorn extract manufactured by the above method contains the following compounds of formula (I) to (III):

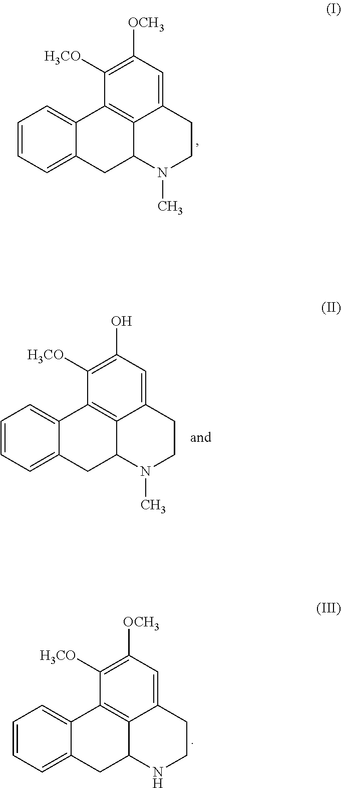 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

The inventors of the present invention found that all of the compounds of formula (I) to formula (III) have the effect of reducing the fat content of adipocyte, and thus, those compounds and the fermentation product of lotus leaf-hawthorn extract comprising those compounds can be manufactured into a pharmaceutical composition or a food composition. Alternatively, those compounds and the fermentation product of lotus leaf-hawthorn extract comprising those compounds can be used in the form of a pharmaceutical composition or a food composition, wherein the pharmaceutical composition can be used for at least one of promoting the lipid-degradation ability of adipocyte, inhibiting the lipid-forming ability of adipocyte and promoting weight loss, and the food composition can be used for making the formation of body fat difficult.

The pharmaceutical composition provided in accordance with the present invention may be administered to a subject in need systemically or topically, and may be delivered by various drug delivery systems (DDSs), such as an oral drug delivery system, injectable drug delivery system, etc. For example, to enhance bioavailability, control drug release speed, precisely target the lesion and reduce side effects, the pharmaceutical composition may be delivered by liposomes, microcapsules, nanoparticles, microneedles, but is not limited thereto.

Depending on the desired purpose(s), the pharmaceutical composition of the present invention could be provided in any suitable form without particular limitations. For example, the pharmaceutical composition could be provided in a form suitable for administering to a subject in need orally or parenterally (such as an intraperitoneal injection, muscle injection, intravenous injection, and subcutaneous injection), but is not limited thereto. Depending on the form and purpose(s), a suitable carrier could be chosen and used to provide the pharmaceutical composition. Examples of the carrier include excipients, diluents, auxiliaries, stabilizers, absorbent retarders, disintegrating agents, hydrotropic agents, emulsifiers, antioxidants, adhesives, binders, tackifiers, dispersants, suspending agents, lubricants, hygroscopic agents, solvents, chelating agents, gelling agents, etc.

In the form for oral administration, the pharmaceutical composition of the present invention can comprise any pharmaceutically acceptable carrier that will not adversely affect the desired effects of the active ingredient(s) (i.e., any one of the compounds of formula (I) to formula (III) and/or the fermentation product of lotus leaf-hawthorn extract). Examples of the suitable carrier include, but are not limited to, water, saline, dextrose, glycerol, ethanol or its analogs, cellulose, starch, sugar bentonite, and combinations thereof. The pharmaceutical composition could be provided by any suitable method in any form suitable for oral administration, such as in the form of a troche (e.g., dragee), a pill, a tablet, a capsule, a granule, a pulvis, a fluidextract, a solution, a syrup, a suspension, or a tincture, but is not limited thereto.

As for the form of injection or drip suitable for intraperitoneal, muscle, intravenous or subcutaneous injection administration, the pharmaceutical composition may comprise one or more ingredient(s), such as an isotonic solution, a salt-buffered saline (e.g., phosphate-buffered saline or citrate-buffered saline), a hydrotropic agent, an emulsifier, a sugar solution, and other carriers to provide the pharmaceutical composition as an intravenous infusion, an emulsified intravenous infusion, a powder for injection, a suspension for injection, or a powder suspension for injection. Alternatively, the pharmaceutical composition may be prepared as a pre-injection solid. The pre-injection solid can be provided in a form which is soluble in other solutions or suspensions, or in an emulsifiable form. A desired injection is provided by dissolving the pre-injection solid in other solutions or suspensions, or emulsifying it prior to being administered to a subject in need.

Depending on the needs, age, body weight and health conditions of the subject, the pharmaceutical composition provided in accordance with the present invention could be administered at various administration frequencies, such as once a day, multiple times a day, or once every few days, etc. In addition, the amount of the compounds of formula (I) to formula (III) and/or the fermentation product of lotus leaf-hawthorn extract in the composition could be adjusted depending on the requirements of practical application. In general, when used in the form of a pharmaceutical composition, the recommended dosage for an adult is about 5 g/day to 15 g/day of fermentation product of lotus leaf-hawthorn extract; or, about 10 ppm/day to 100 ppm/day of at least one of the compounds of formula (I) to formula (III). Additionally, the pharmaceutical composition provided in accordance with the present invention could further comprise one or more other active ingredients (e.g., obesity drugs, catechin and β-glucan), or be used in combination with a medicament comprising one or more other active ingredients to further enhance the effects of the pharmaceutical composition, or to increase the application flexibility and adaptability of the preparation thus provided, as long as the other active ingredients will not adversely affect the desired effects of the active ingredient(s) of the present invention (i.e., any one of the compounds of formula (I) to formula (III) and/or the fermentation product of lotus leaf-hawthorn extract).

Optionally, the pharmaceutical composition and the food composition provided in accordance with the present invention could further comprise a suitable amount of additives, such as a flavoring agent, a toner, or a coloring agent for enhancing the palatability and the visual perception of the pharmaceutical composition or the food composition, and/or a buffer, a conservative, a preservative, an antibacterial agent, or an antifungal agent for improving the stability and storability of the pharmaceutical composition or the food composition.

The food composition of the present invention can comprise any edible carrier that will not adversely affect the desired effects of the active ingredient(s) (i.e., any one of the compounds of formula (I) to formula (III) and/or the fermentation product of lotus leaf-hawthorn extract). Examples of suitable carriers include, but are not limited to, water, saline, dextrose, glycerol, ethanol or its analogs, cellulose, starch, sugar bentonite, and combinations thereof.

The food composition provided in accordance with the present invention can be a health food, a dietary supplement, a functional food, a nutritional supplement or a special nutritional food, and can be provided as dairy products, meat products, breadstuff, pasta, cookies, troche, capsules, fruit juices, teas, sport beverages, nutritional beverages, etc., but is not limited thereto. Preferably, the food composition provided in accordance with the present invention is a health food.

Depending on the age, body weight and health condition of the subject, the health food, dietary supplement, functional food, nutritional supplement and special nutritional food provided in accordance with the present invention could be taken at various frequencies, such as once a day, several times a day or once every few days, etc. The amount of the compounds of formula (I) to formula (III) and/or the fermentation product of lotus leaf-hawthorn extract in the health food, dietary supplement, functional food, nutritional supplement and special nutritional food provided in accordance with the present invention could be adjusted, for example, to an amount such that it should be taken daily, depending on the specific population. In general, when used in the form of a food composition, the recommended dosage for an adult is about 5 g/day to 15 g/day of the fermentation product of lotus leaf-hawthorn extract; or, about 10 ppm/day to 200 ppm/day of at least one of the compounds of formula (I) to formula (III).

The recommended daily dosage, use standards and use conditions for a specific population (e.g., patients with diabetes, patients with hyperlipidemia, and pregnant women), or the recommendations for a use in combination with another food product or medicament can be indicated on the exterior package of the health food, dietary supplement, functional food, nutritional supplement and/or special nutritional food provided in accordance with the present invention. Thus, it is safe for the user to take the health food, dietary supplement, functional food, nutritional supplement and/or special nutritional food by him- or herself without instructions of a doctor, pharmacist, or related executive.

As set forth above, the present invention also provides a method for at least one of promoting the lipid-degradation ability of adipocyte, inhibiting the lipid-forming ability of adipocyte, making the formation of body fat difficult and promoting weight loss, the method comprising administering to a subject in need an effective amount of an active ingredient, wherein the active ingredient is at least one of the compounds of formula (I) to formula (III). The aforementioned term “a subject in need” refers to a subject having excessive body fat, a high BMI value, obesity, or the desire to improve his/her appearances. Additionally, since obesity is closely related to conditions such as diabetes, fatty liver, hyperlipidemia, hypertension, heart disease, stroke and cancer, the aforementioned subject can also refer to a subject wanting to prevent the foregoing diseases. In the above method, the active ingredient could be administered to the subject in need in the form of a pharmaceutical composition or a food composition as described above, wherein the administration type, administration route, administration form, administration frequency and uses of the pharmaceutical composition and the food composition are all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

The sources of the materials used in the following Examples are as follows:
1. Lotus leaf: purchased from China.
2. Hawthorn: purchased from China.
3. Saccharomyces cerevisiae BCRC 20271: purchased from Bioresource Collection and Research Center (BCRC).
4. Lactobacillus plantarum BCRC 910760: purchased from Bioresource Collection and Research Center.
5. Acetobacter aceti BCRC 11688: purchased from Bioresource Collection and Research Center.
6. MEM-α medium: purchased from Gibco.
7. Fetal Bovine Serum (FBS): purchased from Gibco.
8. Penicillin/Streptomycin: purchased from Gibco.
9. Phosphate buffered saline (PBS): purchased from Gibco.
10. Oil-red O dye: purchased from Sigma company.
11. Formaldehyde: purchased from Echo chemical company.
12. Isopropanol: purchased from Echo chemical company.
13. n-butanol: purchased from Merck Taiwan.
14. Diaion HP-20 resin: purchased from Mitsubishi chemical company.
15. Sephadex LH-20 gel: purchased from Amersham Biosciences company.
16. Thin layer chromatography plate (silica gel 60 F₂₅₄, 0.25 mm/RP-18 F_{254S}, 0.25 mm): purchased from Merck Germany.
17. Medium pressure liquid chromatography (MPLC): CombiFlash® Rf, purchased from Teledyne ISCO company.
18. High Performance Liquid Chromatography (HPLC):
   (i) Pump system: Hitachi L-2310 series pump;
   (ii) Detector: Hitachi L-2420 UV-VIS detector, in which the detecting wavelength used was 200˜380 nm;
   (iii) Data analyzing software: D-2000 Elite software;
   (iv) Analyzing column: Discovery® HS C18 (SUPELCO, 250×4.6 mm, 5 μm);
   (v) Analyzing column: Mightysil RP-18 GP 250 (Kanto, 250×4.6 mm, 5 μm);
   (vi) Semipreparative column: Discovery® HS C18 (SUPELCO, 250×10.0 mm, 5 μm);
   (vii) Preparative column: Discovery® HS C18 (SUPELCO, 250×21.0 mm, 5 μm).
19. UV light: UVP UVGL-25, in which the wavelengths used were 254 nm and 365 nm.
20. Nuclear Magnetic Resonance Spectrometer (NMR): 1D and 2D spectrum used 400 MHz Varian 400 FT-NMR, wherein the chemical shift was represented as 6, the unit being ppm.
21. Mass Spectrometer (MS): LTQ-FT, using Bruker amaZon SL system for measuring, the unit being m/z.
22. Mice mesenchymal cell line OP9 (ATCC CRL-2749): purchased from American Type Culture Collection (ATCC).

### PREPARATION EXAMPLES

### A. Preparation of the Fermentation Product of Lotus Leaf-Hawthorn Extract

A-1. Lotus leaf and hawthorn were taken and made into lotus leaf pieces and hawthorn pieces by cutting and grinding. The lotus leaf pieces and hawthorn pieces were then mixed at a weight ratio ranging from 1:1 to 3:1 to provide a mixture. Thereafter, the mixture and purified water were mixed at a weight ratio ranging from 2:85 to 4:65, followed by extraction at 50° C. to 100° C. for 0.5 to 3 hours to provide a lotus leaf-hawthorn extract. The lotus leaf-hawthorn extract was cooled down to room temperature before the following fermentation process.

A-2. The lotus leaf-hawthorn extract provided in A-1 was taken, and Saccharomyces cerevisiae BCRC 20271 and Lactobacillus plantarum BCRC 910760 were simultaneously added therein. The extract was then subjected to a fermentation process at 25° C. to 35° C. for 1 to 3 days to provide an intermediate fermentation product. The added amount of Saccharomyces cerevisiae BCRC 20271 was 0.01 wt. % to 0.5 wt. % of the lotus leaf-hawthorn extract. The added amount of Lactobacillus plantarum BCRC 910760 was 0.01 wt. % to 0.25 wt. % of the lotus leaf-hawthorn extract.

A-3. The intermediate fermentation product provided in A-2 was taken, and Acetobacter aceti BCRC 11688 was added therein. The intermediate fermentation product was then subjected to a fermentation process at 25° C. to 35° C. for 5 to 15 days to provide a fermentation product of lotus leaf-hawthorn extract. The added amount of Acetobacter aceti BCRC 11688 was 1 wt. % to 20 wt. % of the intermediate fermentation product. The fermentation product provided by the above steps had a sugar degree ranging from 3° to 10°, a pH value ranging from 2 to 4, and an alcohol concentration of 3% to 15% (w/v).

A-4. The fermentation product of lotus leaf-hawthorn extract provided in A-3 was taken and subjected to a vacuum concentration at 45° C. to 70° C. to provide a concentrated fermentation product. Thereafter, the concentrated fermentation product was filtered by using a mesh screen with 200 to 400 meshes to remove the solid residues. Finally, isomaltooligosaccharides was added to the fermentation product treated by the aforementioned processes in an amount of about 40% to about 70% (w/w), and then the fermentation product was sterilized at 90° C. to 130° C. for 70 minutes to 120 minutes to provide a beverage comprising the fermentation product of lotus leaf-hawthorn extract.

### B. Preparation of Compounds of Formula (I) to Formula (III)

B-1. 10 L of the fermentation product of lotus leaf-hawthorn extract provided in A-3 was taken and subjected to a liquid-liquid extraction with the use of n-butanol and water (ratio: 1:1). The extraction was repeated for three times, and the three extracts thus obtained were combined and dried by using vacuum concentration to provide a n-butanol layer extract (58.4 g) and a water layer extract (520 g).

B-2. The n-butanol layer extract provided in B-1 was taken and isolated with the use of Diaion HP-20 resin as the material for chromatographic analysis. The eluant used in the isolation was started with purified water, and then methanol was added and the ratio of methanol in the water was increased to lower the polarity of the eluant. After the aforementioned isolation step, 3 fractions (F1 to F3) were obtained. The second fraction (F2, 15.7 g) was taken and further isolated with the use of Sephadex LH-20 gel as the material for column chromatography, with methanol being the eluant. Thereafter, the fractions were combined with the use of thin layer chromatography plate. 5 subfractions (F2-1 to F2-5) were obtained, and then subfraction F2-1 was taken and further isolated with the use of Medium pressure liquid chromatography (MPLC), wherein the eluant was a solvent provided by mixing water and methanol. Finally, the fractions were combined with the use of thin layer chromatography plate, and 7 subfractions (F2-1-1 to F2-1-7) were thus obtained.

B-3. The subfraction F2-1-1 provided in B-2 was taken and purified with the use of HPLC and a solvent provided by mixing methanol and water (1:1) as the mobile phase. Finally, the compound of formula (I) (20.0 mg), the compound of formula (II) (5.5 mg), and the compound of formula (III) (3.0 mg) were obtained.

B-4. The compounds of formula (I) to formula (III) provided in B-3 were taken and analyzed by using Nuclear Magnetic Resonance Spectrometer. The structures of those compounds were identified and are shown below:

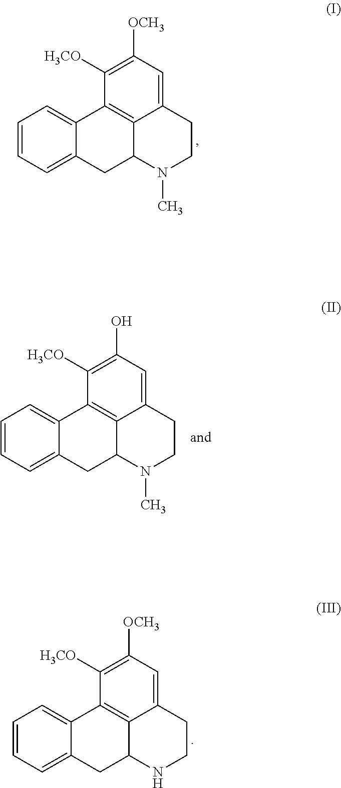 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

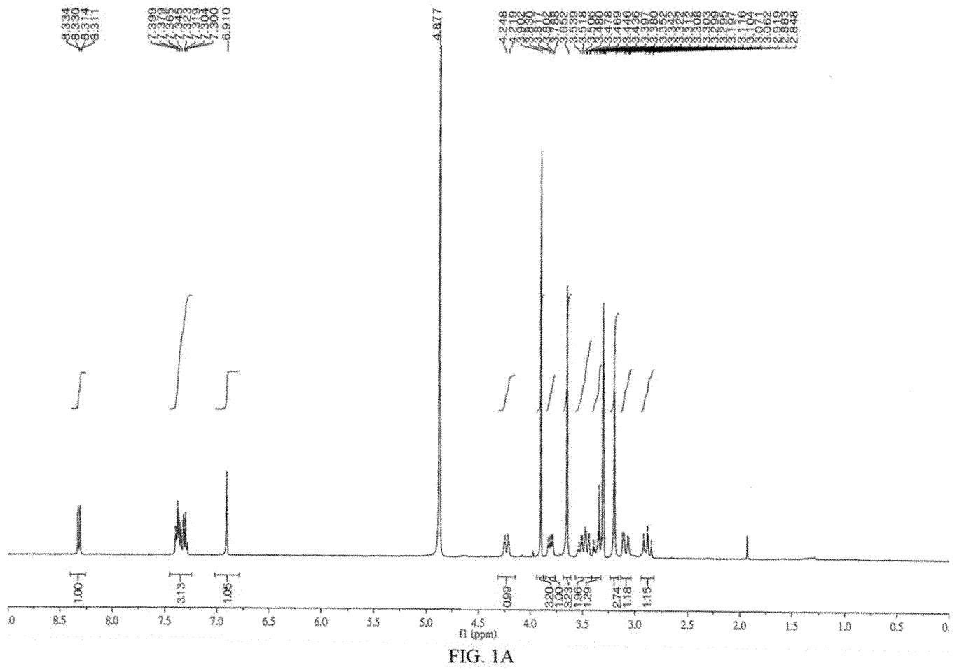

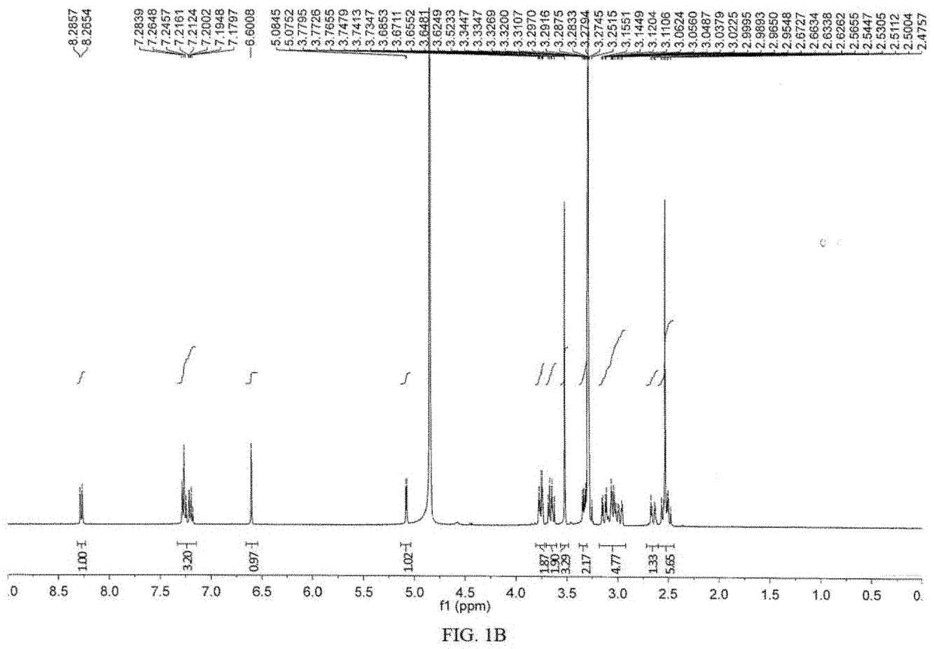

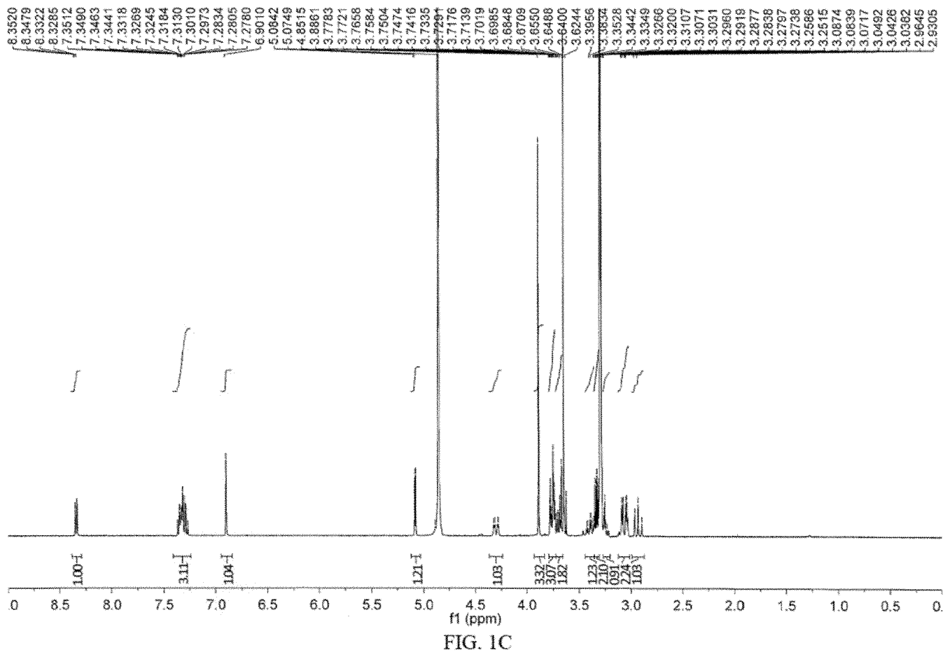

The NMR spectrums of the above compounds are shown in FIGS. 1A to 1C, respectively.

### C. Cell Culture

Mice mesenchymal cell line OP9 cells were seeded in a 24-well plate (8×10⁴ cells/well), wherein each well contained 500 μL preadipocyte expansion medium (i.e., 90% MEM-α medium added with 20% FBS and 1% Penicillin/Streptomycin). The cells were cultured at 37° C. for 7 days, wherein the medium was replaced with an adipocyte differentiation medium (i.e., 90% MEM-α medium added with 20% FBS and 1% Penicillin/Streptomycin) every 3 days. After 7 days, a microscope (ZEISS Axio Vert.A1) was used to observe the oil drops in the cells to confirm that those cells were totally differentiated into adipocytes.

### D. Preparation of Oil-Red O Dye

Oil-red O dye was completely dissolved into 100% isopropanol to prepare a 30 mg/mL oil-red O stock solution. Prior to usage, the stock solution was diluted to a concentration of 18 mg/mL with double distillation water, and filtered with a 0.22 μm filter membrane. A useable oil-red O dye was thus provided.

### Cell Experiments

### Example 1: The Effect of the Fermentation Product of Lotus Leaf-Hawthorn Extract on Reducing the Fat Content of Adipocyte

Adipocytes provided in [Preparation example C] were taken and divided into three groups, and were separately cultured in the following medium at 37° C. for 7 days (the media were replaced every 3 days):
1. Group I: adipocytes differentiation medium (500 μL in total).
2. Group II: adipocytes differentiation medium containing 5% (w/w) lotus leaf-hawthorn extract provided in [Preparation example A-1] (500 μL in total).
3. Group III: adipocytes differentiation medium containing 5% (w/w) fermentation product of lotus leaf-hawthorn extract provided in [Preparation example A-3] (500 μL in total).

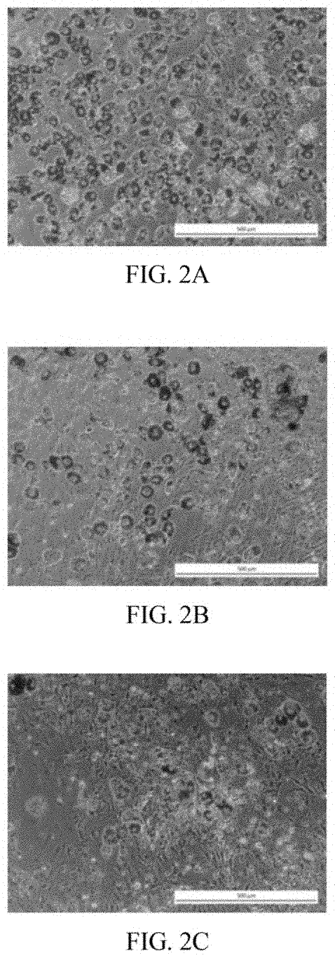

Thereafter, the cells provided by groups I to III were treated by the following dying and quantifying steps: removing the medium and washing the cells with PBS solution, fixing the cells with 10% formaldehyde at room temperature for 30 minutes, washing the fixed cells with PBS solution once, and rinsing the cell with 60% isopropanol for 1 minute. Thereafter, the isopropanol was removed, and the cells after the above treatments were dyed with the oil-red O dye provided in [Preparation example D] for 1 hour. Next, the oil-red O dye was removed, and the cells were decolorized with 60% isopropanol for 5 seconds. Then, after washing the dyed cells with PBS solution, a microscope (ZEISS Axio Vert.A1) was used to observe the cells and take photos thereof. The photos thus obtained are shown in FIGS. 2A to 2C.

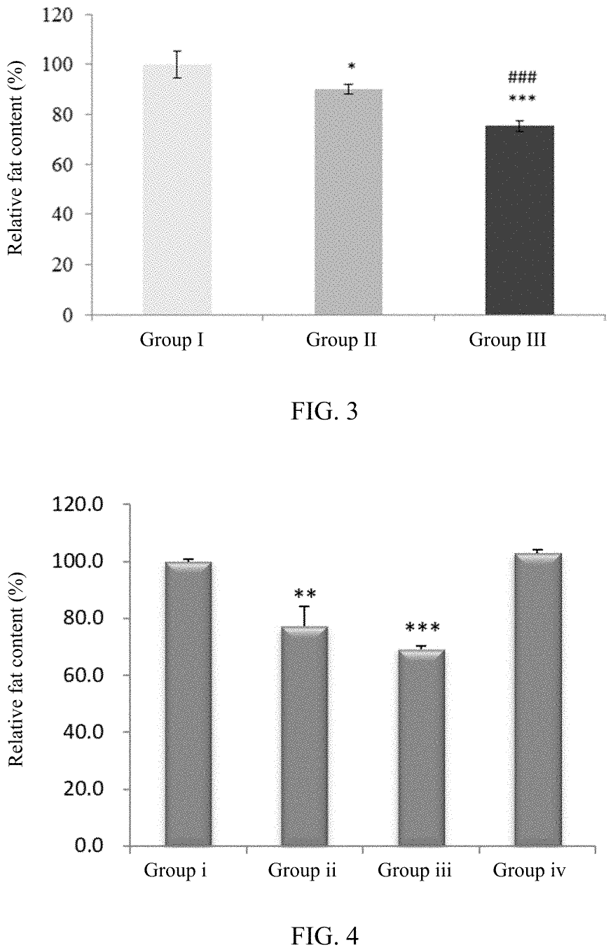

The above cells were taken, and 100% isopropanol was used to dissolve the dye in those cells (for 10 minutes). 100 μL isopropanol that contained the dissolved dye was moved to a 96-well plate. An ELISA reader (BioTek) was then used to measure the absorbance at 510 nm to quantify the dissolved dye and measure the fat content of the cells. Finally, the result of the control group (i.e., cells cultured with the medium of group I) was used as a basis to calculate the relative fat content of the other groups. The results are shown in FIG. 3.

As shown in FIGS. 2A to 2C, as compared to the control group and the lotus leaf-hawthorn extract group, the area in the cells treated with the fermentation product of lotus leaf-hawthorn extract of the present invention (i.e., cells cultured with the medium of group III) that was dyed by the oil-red O dye was significantly reduced. Moreover, as shown in FIG. 3, as compared to the control group and the lotus leaf-hawthorn extract group, the fat content of the cells treated with the fermentation product of lotus leaf-hawthorn extract of the present invention (i.e., cells cultured with the medium of group III) was significantly reduced. These results indicate that the fermentation product of lotus leaf-hawthorn extract of the present invention can effectively reduce the fat content of adipocyte, and thus can be used for at least one of promoting the lipid-degradation ability of adipocyte, inhibiting the lipid-forming ability of adipocyte, making the formation of body fat difficult and promoting weight loss.

### Example 2: The Effects of Different Extraction Layers of the Fermentation Product of Lotus Leaf-Hawthorn Extract on Reducing the Fat Content of Adipocyte

Adipocytes provided in [Preparation example C] were taken and divided into four groups, and were separately cultured in the following medium at 37° C. for 7 days (the media were replaced every 3 days):
1. Group i: adipocytes differentiation medium (500 μL in total).
2. Group ii: adipocytes differentiation medium containing 5% (w/w) fermentation product of lotus leaf-hawthorn extract provided in [Preparation example A-3] (500 μL in total).
3. Group iii: adipocytes differentiation medium containing 5% (w/w) n-butanol layer extract provided in [Preparation example B-1] (500 μL in total).
4. Group iv: adipocytes differentiation medium containing 5% (w/w) water layer extract provided in [Preparation example B-1] (500 μL in total).

Thereafter, the cells provided by groups i to iv were treated by the dying and quantifying steps as described in the above Example 1. Finally, the result of the control group (i.e., cells cultured with the medium of group i) was used as a basis to calculate the relative fat content of the other groups. The results are shown in FIG. 4.

As shown in FIG. 4, as compared to the control group, the fat contents of the cells treated with the fermentation product of lotus leaf-hawthorn extract of the present invention (i.e., cells cultured with the medium of group ii) and the cells treated with the n-butanol layer extract of the fermentation product of lotus leaf-hawthorn extract (i.e., cells cultured with the medium of group iii) were significantly reduced. However, the fat content of the cells treated with the water layer extract of the fermentation product of lotus leaf-hawthorn extract (i.e., cells cultured with the medium of group iv) was equal to that of the control group. These results indicate that the effect of the fermentation product of lotus leaf-hawthorn extract of the present invention on reducing the fat content of adipocyte is mainly provided by the ingredients in the n-butanol layer extract.

### Example 3: Effect of Compounds of Formula (I) to Formula (III) on Reducing the Fat Content of Adipocyte

Adipocytes provided in [Preparation example C] were taken and divided into four groups, and were separately cultured in the following medium at 37° C. for 7 days (the media were replaced every 3 days):
1. Group A: adipocytes differentiation medium (500 μL in total).
2. Group B: adipocytes differentiation medium containing 20 μg/mL of the compound of formula (I) provided in [Preparation example B-3] (500 μL in total).
3. Group C: adipocytes differentiation medium containing 20 μg/mL of the compound of formula (II) provided in [Preparation example B-3] (500 μL in total).
4. Group D: adipocytes differentiation medium containing 20 μg/mL of the compound of formula (III) provided in [Preparation example B-3] (500 μL in total).

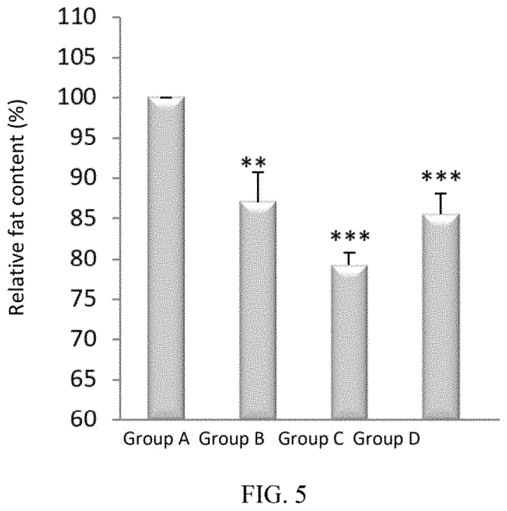

Thereafter, the cells provided by groups A to D were treated by the dying and quantifying steps as described in the above Example 1. Finally, the result of the control group (i.e., cells cultured with the medium of group A) was used as a basis to calculate the relative fat content of the other groups. The results are shown in FIG. 5.

As shown in FIG. 5, as compared to the control group, the fat contents of the cells treated with the compounds of formula (I) to formula (III) (i.e., cells cultured with the medium of groups B to D) were significantly reduced. These results indicate that the compounds of formula (I) to formula (III) of the present invention can effectively reduce the fat content of adipocyte, and thus, those compounds can be used for at least one of promoting the lipid-degradation ability of adipocyte, inhibiting the lipid-forming ability of adipocyte, making the formation of body fat difficult and promoting weight loss.

### Example 4: The Change of the Contents of the Compounds of Formula (I) to Formula (III) in the Lotus Leaf-Hawthorn Extract Before and after Conducting the Fermentation Process

The lotus leaf-hawthorn extract provided in [Preparation example A-1] and the fermentation product of lotus leaf-hawthorn extract provided in [Preparation example A-3] were taken and respectively formulated into sample solutions that had a concentration of 20 mg/mL. Thereafter, 10 μL of each sample solution were taken, and the ingredients in the sample solutions were analyzed using HPLC. The analyzing column used in the HPLC was Mightysil RP-18 GP 250 (250×10 mm, 5 μm), the detecting wavelength was 280 nm, the elution rate was 1.0 mL/min, and the composition of the eluant is shown in the following Table 1:

TABLE 1

| Time (min) | Mobile phase A (%) | Mobile phase B (%) |
|---|---|---|
| 0 | 98 | 2 |
| 10 | 98 | 2 |
| 40 | 30 | 70 |
| 50 | 0 | 100 |
| 60 | 0 | 100 |
| 62 | 98 | 2 |
| 70 | 98 | 2 |

*A: water + 0.1% formic acid; B: methano1+ 0.1% formic acid

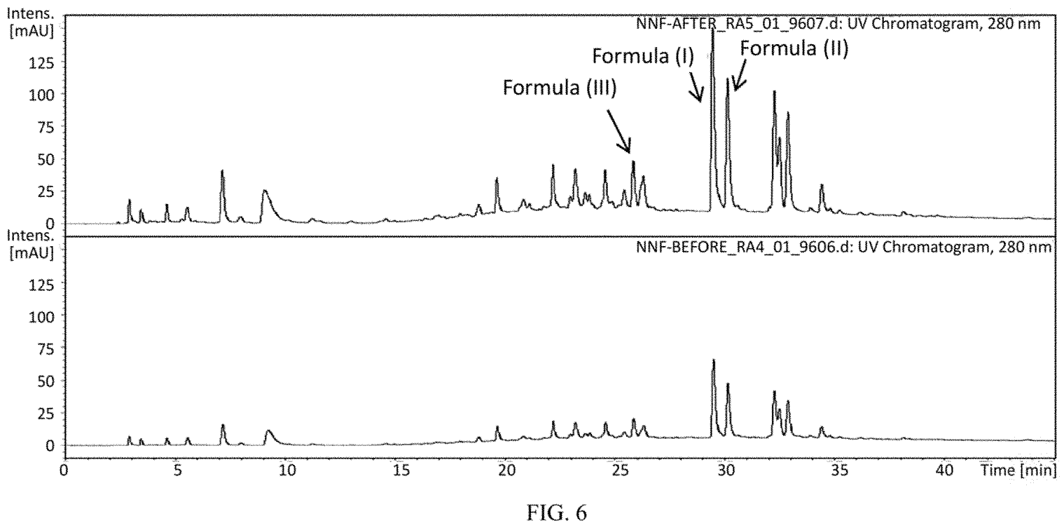

The HPLC fingerprints obtained by conducting the above steps are shown in FIG. 6. As shown in FIG. 6, as compared to the lotus leaf-hawthorn extract, the contents of the compounds of formula (I) to formula (III) in the fermentation product of lotus leaf-hawthorn extract prepared by the fermentation method of the present invention were higher. These results indicate that the method of the present invention for manufacturing the fermentation product of lotus leaf-hawthorn extract can effectively increase the contents of the active ingredients contained in the lotus leaf-hawthorn extract.

### Human Experiments

### Example 5: Effect of the Fermentation Product of Lotus Leaf-Hawthorn Extract on Promoting Weight Loss

(5-1) obtaining the data of week 0: volunteers (10 in total, with ages ranging from 20 to 60, BMI values greater than 24, a body fat percentage greater than 25% for males, and a body fat percentage greater than 30% for females) were recruited. The subjects' body weights, waistlines, body mass indexes (BMI), body fat percentages and trunk fat percentages were measured with a body composition monitor (TANITA BC-601FS). The subjects were requested to evaluate the level of obesity of themselves (the evaluation items include: high body weight, high body fat percentage, large waistline, big hip size, and protruding tummy). The above data were used in the following experiments.

(5-2) The subjects were requested to drink the beverage containing the fermentation product of lotus leaf-hawthorn extract provided in [Preparation example A-4] (which contained 10 g of the fermentation product of lotus leaf-hawthorn extract) every day for four weeks. During the four weeks, the diet of all of the subjects was not adjusted.

(5-3) obtaining the data of week 4: The subjects' body weights, waistlines, body mass indexes (BMI), body fat percentages and trunk fat percentages were measured with a body composition monitor (TANITA BC-601FS), and the subjects were requested to evaluate the level of obesity of themselves (the evaluation items include: high body weight, high body fat percentage, large waistline, big hip size, and protruding tummy).

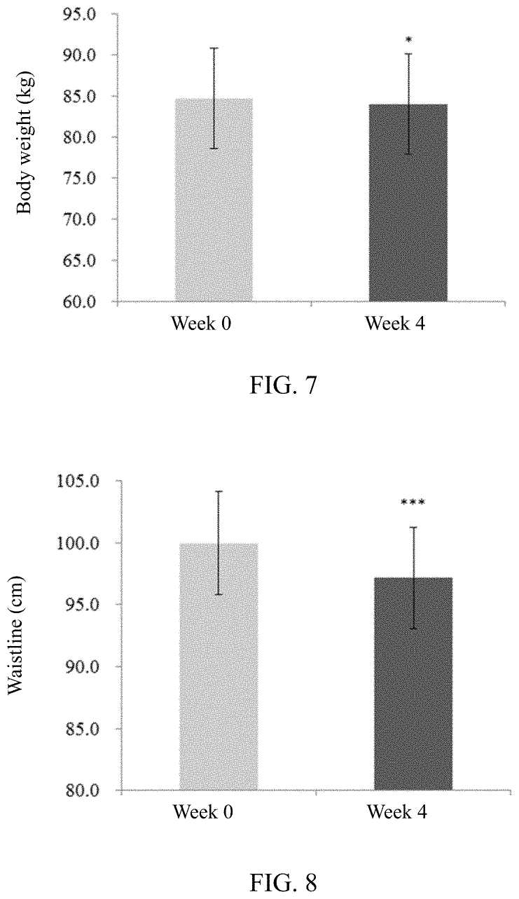

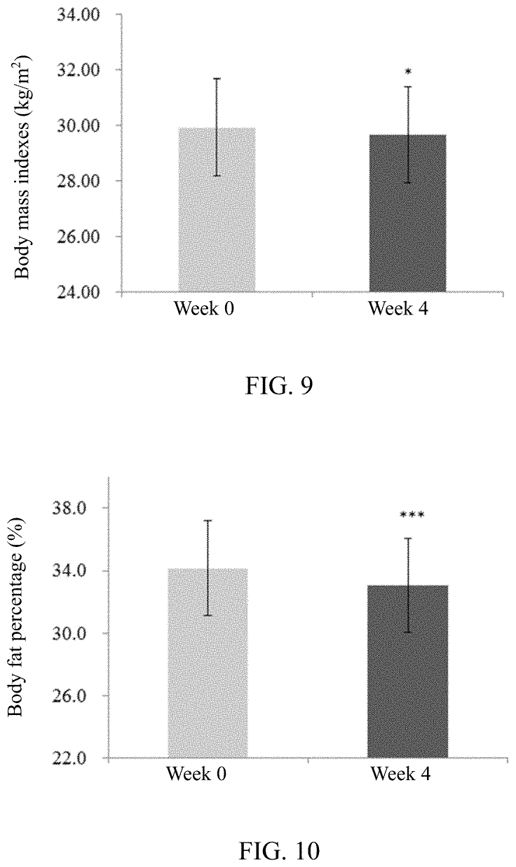

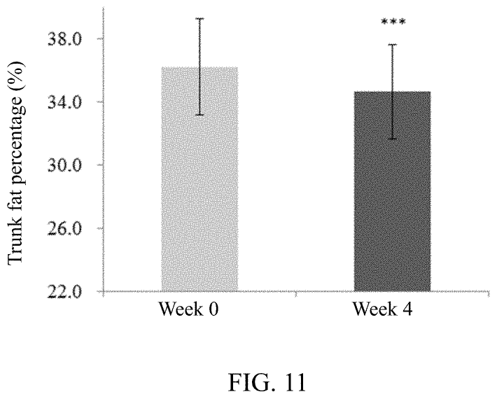

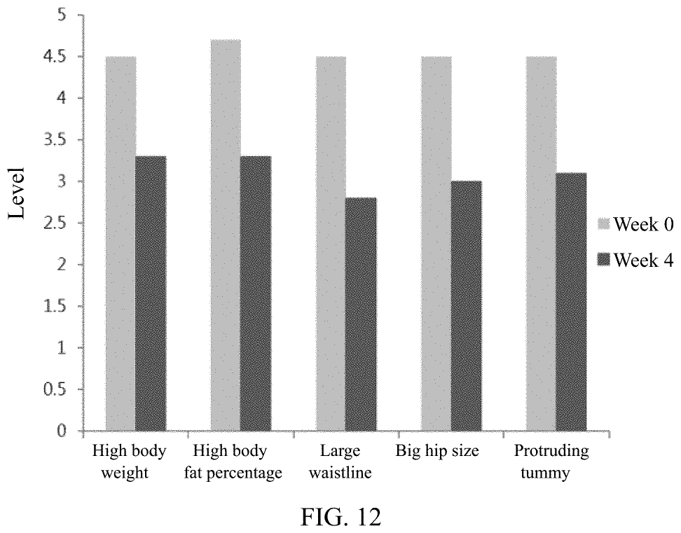

(5-4) Data obtained at week 0 and week 4 were analyzed. The body weights, waistlines, body mass indexes (BMI), body fat percentages and trunk fat percentages of the subjects before taking the fermentation product of lotus leaf-hawthorn extract (i.e., week 0) and after taking the fermentation product of lotus leaf-hawthorn extract (i.e., week 4) are respectively shown in FIGS. 7 to 11. In addition, the evaluation results made by the subjects for the level of obesity of themselves are shown in FIG. 12.

As shown in FIGS. 7 to 11, as compared to week 0, the body weights, waistlines, body mass indexes (BMI), body fat percentages and trunk fat percentages of the subjects at week 4 were all reduced, wherein the changes in the waistlines, body fat percentages and trunk fat percentages were the most significant. As shown in FIG. 12, after taking the fermentation product of lotus leaf-hawthorn extract for 4 weeks, the evaluation scores made by the subjects for the level of obesity of themselves were also significantly reduced. These results indicate that the fermentation product of lotus leaf-hawthorn extract of the present invention is useful for promoting weight loss.

In the above examples, all the data were calculated and analyzed by using Excel software with the results being shown in the form of average±standard deviation. The statistical significance was determined by using Student's t-test, wherein in the appended Figures, * indicates that, as compared to the control group, the p value<0.05; ** indicates that, as compared to the control group, the p value<0.01; *** indicates that, as compared to the control group, the p value<0.001; ### indicates that, as compared to the lotus leaf-hawthorn extract group (i.e., group II of Example 1), the p value<0.001.

As shown in the above Examples, the fermentation product of lotus leaf-hawthorn extract of the present invention and the compounds of formula (I) to formula (III) are effective in reducing the fat content of adipocyte, and thus, can be used for at least one of promoting the lipid-degradation ability of adipocyte, inhibiting the lipid-forming ability of adipocyte, making the formation of body fat difficult and promoting weight loss.

1. A method for at least one of promoting the lipid-degradation ability of adipocyte, inhibiting the lipid-forming ability of adipocyte, making the formation of body fat difficult and promoting weight loss, the method comprising administering to a subject in need an effective amount of an active ingredient, wherein the active ingredient is at least one of the compounds of formula (I) to formula (III):

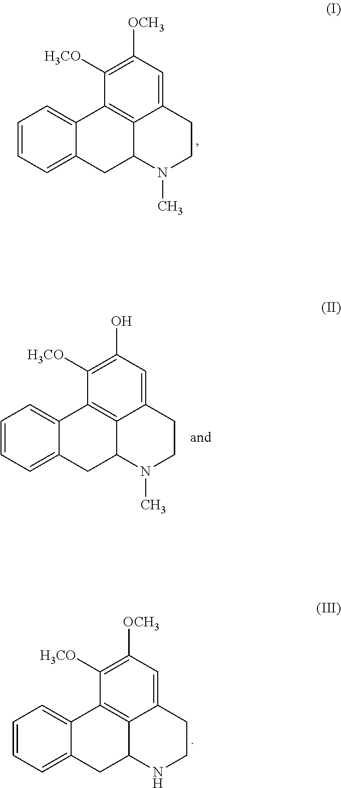 [SMILES: COc1cc2c3c(c1OC)-c1ccccc1CC3N(C)CC2 ; COc1c(O)cc2c3c1-c1ccccc1CC3N(C)CC2 ; COc1cc2c3c(c1OC)-c1ccccc1CC3NCC2]

2. The method as claimed in claim 1, wherein the active ingredient is provided in the form of a fermentation product of an extract.

3. The method as claimed in claim 2, wherein the extract is a lotus leaf-hawthorn extract.

4. The method as claimed in claim 1, which is for at least one of promoting the lipid-degradation ability of adipocyte, inhibiting the lipid-forming ability of adipocyte and promoting weight loss, wherein the active ingredient is administered to the subject in the form of a pharmaceutical composition.

5. The method as claimed in claim 1, which is for making the formation of body fat difficult, wherein the active ingredient is administered to the subject in the form of a food composition.

6. A composition for at least one of promoting the lipid-degradation ability of adipocyte, inhibiting the lipid-forming ability of adipocyte, making the formation of body fat difficult and promoting weight loss, the composition comprising a fermentation product of lotus leaf-hawthorn extract.

7. The composition as claimed in claim 6, wherein the fermentation product is provided by the following steps:
(a) mixing lotus leaf and hawthorn to provide a mixture;
(b) extracting the mixture to provide an extract;
(c) fermenting the extract by using Saccharomyces cerevisiae and Lactobacillus sp. strains to provide an intermediate fermentation product; and
(d) fermenting the intermediate fermentation product by using an Acetobacter sp. strain to provide a fermentation product of lotus leaf-hawthorn extract.

8. The composition as claimed in claim 7, wherein the weight ratio of lotus leaf and hawthorn in step (a) is 1:1 to 3:1.

9. The composition as claimed in claim 7, wherein the extraction in step (b) is performed by using water that optionally contains saccharide.

10. The composition as claimed in claim 8, wherein the extraction in step (b) is performed by using water that optionally contains saccharide.

11. A method for manufacturing a fermentation product of lotus leaf-hawthorn extract, the method comprising the following steps:
(a) mixing lotus leaf and hawthorn to provide a mixture;
(b) extracting the mixture to provide an extract;
(c) fermenting the extract by using Saccharomyces cerevisiae and Lactobacillus sp. strains to provide an intermediate fermentation product; and
(d) fermenting the intermediate fermentation product by using an Acetobacter sp. strain to provide a fermentation product of lotus leaf-hawthorn extract.

12. The method as claimed in claim 11, wherein the weight ratio of lotus leaf and hawthorn in step (a) is 1:1 to 3:1.

13. The method as claimed in claim 11, wherein the extraction in step (b) is performed by using water that optionally contains saccharide.

14. The method as claimed in claim 12, wherein the extraction in step (b) is performed by using water that optionally contains saccharide.
